(19) United States
(12) Patent Application Publication
(10) Pub. No.: US 2025/0057492 A1
(43) Pub. Date: Feb. 20, 2025

(54) SYSTEM AND METHOD FOR MOBILE IMAGING

(71) Applicant: Medtronic Navigation, Inc., Lafayette, CO (US)

(72) Inventors: David Adams Garlow, Lynnfield, MA (US); John T. Hickey, Merrimack, NH (US)

(21) Appl. No.: 18/798,935

(22) Filed: Aug. 9, 2024

Related U.S. Application Data
(60) Provisional application No. 63/519,697, filed on Aug. 15, 2023.

(51) Int. Cl. A61B 6/00; A61B 6/04

(52) U.S. Cl. CPC A61B 6/4405; A61B 6/0407

(57) ABSTRACT

A mobility system for an imaging system is disclosed. The imaging system is operable to acquire and/or generate image data at positions relative to a subject. The imaging system includes a drive system configured to move the imaging system.

### CROSS-REFERENCE TO RELATED APPLICATION

This application claims the benefit of and priority to U.S. Provisional Patent Application No. 63/519,697 filed Aug. 15, 2023, the entire disclosure of which is incorporated by reference herein.

### FIELD

The subject disclosure is related to an imaging system, and particularly a mobile imaging system.

### BACKGROUND

This section provides background information related to the present disclosure which is not necessarily prior art.

Imaging systems generally include integrated patient supports that are used during an imaging procedure. Generally known imaging systems include the BodyTom® CT Imaging System sold by Neurologica Corp. and the Airo® CT Imaging System sold by Brain Lab. These imaging systems include patient supports that are custom designed to hold the patient and provide a track for rigid movement of the imaging system relative to patient support. Imaging systems may further include bases that are fixed in place. The generally known imaging systems may, therefore, include limited mobility.

### SUMMARY

This section provides a general summary of the disclosure, and is not a comprehensive disclosure of its full scope or all of its features.

A system for acquiring image data of a subject, also referred to as an imaging system, is disclosed. The imaging system may acquire image data that is used to generate images of various types. The images may include reconstructed three-dimensional images, two-dimensional images, or other appropriate image types. In various embodiments, the imaging system may be an X-ray scanner, magnetic resonance imager, or a computed tomography scanner. The image data may be two-dimensional (e.g., projection image data) or other appropriate types of image data, such as three-dimensional image data.

The imaging system may further include a mobility feature that allows it to move relative to a subject. In various embodiments, the subject may be positioned on a support, such as a standard and/or generally known radiolucent surgical table such as a STERIS 4085 SURGICAL TABLE sold by Steris plc, having a place of business in Ohio, that is generally located in selected medical facilities. The imaging system is configured to be positioned relative to the subject to acquire image data of the subject in a selected manner to allow reconstruction of images for display of selected images.

In various embodiments, image data may be acquired while the imaging system is moving relative to the subject. For example, the imaging system may rotate in all or a portion of 360 degrees relative to (e.g., around) the subject. The imaging system may, also or in addition to rotation, translate relative to, such as move along a longitudinal axis of, the subject. In moving along the longitudinal axis of the subject and/or transverse to the longitudinal axis, the imaging system may be driven by a drive system that may include selected wheel supports. The wheel supports may include omni-directional wheels, such as mecanum or omni-wheels. The omni-directional wheels generally include at least one rolling portion mounted on an exterior of a wheel. In various embodiments the wheels may include more than one rolling portion, such as one, two, or eight rolling portions. Each rolling portion may be mounted to one or more wheel plates. As discussed herein, the imaging system may move substantially in one or both of an X-axis and a Y-axis direction or rotate relative to the subject. Further, the imaging system may tilt relative to the subject to acquire image data at an angle relative to the longitudinal axis of the subject.

The imaging system may be moved by a manual manipulation of the imaging system. In various embodiments, the imaging system may include a handle that includes one or more sensors that sense a force, such as pressure, from the user to directly move the imaging system relative to the subject. The manual movement of the imaging system may be inclusive or exclusive of other drive or robotic control features of the imaging system. Accordingly, the user may selectively move the imaging system relative to the subject in an efficient and quick manner without pre-planning a movement of the system.

The imaging system may further include controls, such as automatic or robotic controls, that move the imaging system relative to the subject. The imaging system may move with or according to a pre-planned path relative to the subject for acquiring a selected image data collection of the subject. For example, reconstruction of a selected three-dimensional model of a selected portion of the subject may be selected, and the imaging system may be programmed to automatically move relative to the subject to acquire appropriate amount and type of image data for the three-dimensional reconstruction.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an environmental view of an imaging system;

FIG. 2 is a detail view of a drive system;

FIG. 3A is a schematic view of the imaging system in a first position;

FIG. 3B is a schematic view of the imaging system in a second position;

FIG. 4 is a perspective view of a wheel assembly, according to various embodiments;

FIG. 5 is a perspective view of a partially exploded wheel assembly, according to various embodiments;

FIG. 6 is a perspective view of a partial wheel assembly, according to various embodiments;

FIG. 7 is a cross-sectional view of the roller of the wheel assembly of FIG. 5;

FIG. 8 is an exploded view of a roller of a wheel assembly of FIG. 5;

FIG. 9 is a cross-sectional view of a bearing assembly of the roller of FIG. 7 is a cross-sectional view of a roller of a wheel assembly of FIG. 5;

FIG. 10 is a cross-sectional view of a roller of a wheel assembly according to various embodiments;

FIG. 11 is a schematic view of a force applied to the roller of the wheel assembly of FIG. 5;

FIG. 12 is a schematic view of a force applied to the roller of the wheel assembly of FIG. 5;

FIG. 13 is a perspective view of a wheel assembly, according to various embodiments; and

FIG. 14 is a cross-sectional view of the roller of the wheel assembly of FIG. 13.

Corresponding reference numerals indicate corresponding parts throughout the several views of the drawings.

### DETAILED DESCRIPTION

Example embodiments will now be described more fully with reference to the accompanying drawings.

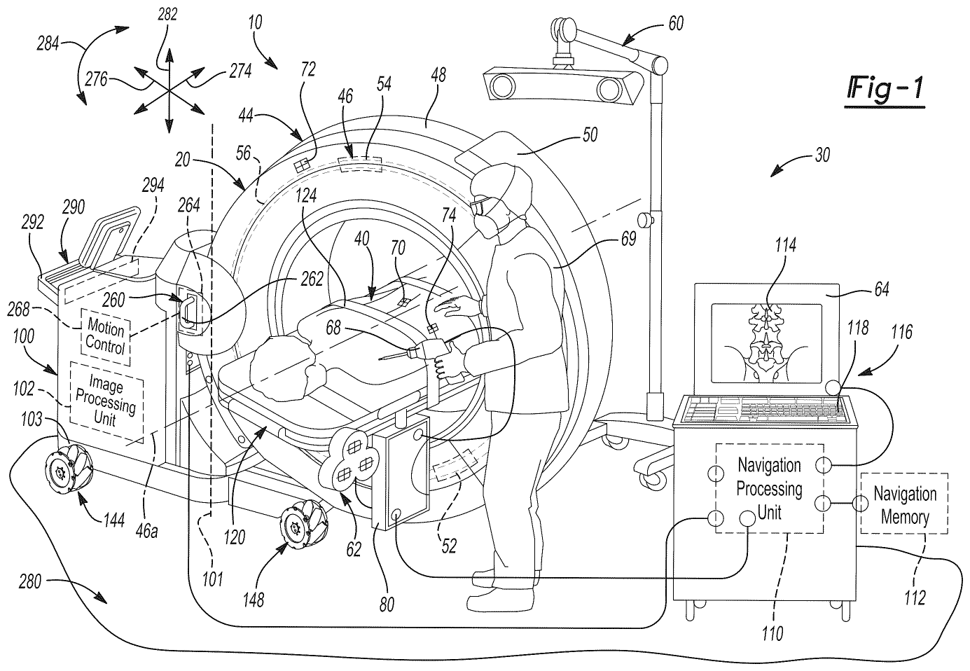

FIG. 1 is a diagram illustrating an overview of an operating theater system 10 that may include an imaging system 20 and a navigation system 30, which can be used for various procedures. The navigation system 30 can be used to track the location of an item, such as an implant or an instrument, relative to a subject, such as a patient 40. It should further be noted that the navigation system 30 may be used to navigate any type of instrument, implant, or delivery system, including: guide wires, arthroscopic systems, orthopedic implants, spinal implants, deep brain stimulation (DBS) probes, etc. Moreover, the instruments may be used to navigate or map any region of the body. The navigation system 30 and the various tracked or navigated items may be used in any appropriate procedure, such as one that is generally minimally invasive or an open procedure.

The navigation system 30 can interface with the imaging system 20 that is used to acquire pre-operative, intra-operative, or post-operative, or real-time image data of the patient 40. It will be understood, however, that any appropriate subject can be imaged and any appropriate procedure may be performed relative to the subject. In the example shown, the imaging system 20 comprises or may include an O-arm® imaging system or device sold by Medtronic Navigation, Inc. having a place of business in Louisville, Colorado, USA. In various embodiments, the imaging system 20 may have a gantry housing 44 that encloses an image data capturing portion 46. The gantry 44 may include a first portion 48 (which may include a generally fixed portion) and a second portion 50 (which may include a moveable portion relative to the first portion 48). The image capturing portion 46 may include an x-ray source or emission portion 52 and an x-ray receiving or image receiving portion (also referred to as a detector that may be operable to detect x-rays) 54 located generally or as practically possible 180 degrees from each other and mounted on a moveable rotor (not illustrated) relative to a track 56 of the image capturing portion 46. The image capturing portion 46 can be operable to rotate 360 degrees around the gantry 44 on or with the rotor during image data acquisition.

In various embodiments, including those discloses further herein, the imaging system 20 may include an imaging system including one or more portions of those imaging systems discloses in U.S. Pat. No. 11,344,268 to Garlow et al., issued May 31, 2022 or U.S. Pat. No. 11,399,784 to Garlow et al., issued Aug. 2, 2022, both incorporated herein by reference. The imaging system may have one or more drive systems, such as one or more motors. The imaging systems may further have one or more wheel assemblies which may include multiple- or omni-directional wheels, such as mecanum wheels.

The image capturing portion 46 may rotate around a central point or axis 46a, allowing image data of the patient 40 to be acquired from multiple directions or in multiple planes. The axis 46a of the imaging system 20 may be aligned or positioned relative to an axis, such as a longitudinal axis, of the patient 40. The imaging system 20 can include all or portions of the systems and methods of those disclosed in U.S. Pat. Nos. 7,188,998; 7,108,421; 7,106,825; 7,001,045; and 6,940,941; all of which are incorporated herein by reference. Other possible imaging systems can include C-arm fluoroscopic imaging systems which can also generate two or three-dimensional views of the patient 40.

The position of the image capturing portion 46 can be precisely known relative to any other portion of the imaging device 20. In addition, as discussed herein, the precise knowledge of the position of the image capturing portion 46 can be used in conjunction with the navigation system 30 having a tracking portion (e.g. an optical tracking system including an optical localizer 60 and/or an electromagnetic (EM) tracking system including an EM localizer 62) to determine the position of the image capturing portion 46 and the image data relative to the tracked subject, such as the patient 40.

Various tracking devices, including those discussed further herein, can be tracked with the navigation system 30 and the information can be used to allow for displaying on a display 64 of a position of an item, e.g. a tool or instrument 68. The instrument may be operated, controlled, and/or held by a user 69. The user 69 may be one or more of a surgeon, nurse, welder, etc. Briefly, tracking devices, such as a patient tracking device 70, an imaging device tracking device 72, and an instrument tracking device 74, allow selected portions of the operating theater to be tracked relative to one another with the appropriate tracking system, including the optical localizer 60 and/or the EM localizer 62. Generally, tracking occurs within a selected reference frame, such as within a patient reference frame.

It will be understood that any of the tracking devices 70, 72, 74 can be optical or EM tracking devices, or both, depending upon the respective tracking system and related tracking localizer used to track the respective tracking devices. It is understood that the tracking devices 70-74 may all be similar or different, and may all be interchangeable but selected or assigned selected purposes during a navigated procedure. It will be further understood that any appropriate tracking system can be used with the navigation system 30. Alterative tracking systems can include radar tracking systems, acoustic tracking systems, ultrasound tracking systems, and the like.

An exemplarily EM tracking system can include the STEALTHSTATION® AXIEM™ Navigation System, sold by Medtronic Navigation, Inc. having a place of business in Louisville, Colorado. Exemplary tracking systems are also disclosed in U.S. Pat. No. 7,751,865, issued Jul. 6, 2010; U.S. Pat. No. 5,913,820, issued Jun. 22, 1999; and U.S. Pat. No. 5,592,939, issued Jan. 14, 1997, all herein incorporated by reference.

Further, for EM tracking systems it may be necessary to provide shielding or distortion compensation systems to shield or compensate for distortions in the EM field generated by the EM localizer 62. Exemplary shielding systems include those in U.S. Pat. No. 7,797,032, issued Sep. 14, 2010 and U.S. Pat. No. 6,747,539, issued Jun. 8, 2004; distortion compensation systems can include those disclosed in U.S. Pat. No. 6,636,757, issued Oct. 21, 2003 and U.S. Pat. No. 9,675,424 issued Jun. 13, 2017, all of which are incorporated herein by reference.

With an EM tracking system, the EM localizer 62 and the various tracking devices can communicate through an EM controller 80. The EM controller can include various amplifiers, filters, electrical isolation, and other systems. The EM controller 80 can also control the coils of the localizer 62 to either emit or receive an EM field for tracking. A wireless communications channel, however, such as that disclosed in U.S. Pat. No. 6,474,341, issued Nov. 5, 2002, herein incorporated by reference, can be used as opposed to being coupled directly to the EM controller 80.

It will be understood that the tracking system may also be or include any appropriate tracking system, including a STEALTHSTATION@ TRIA®, TREON®, and/or S7 ™ Navigation System having an optical localizer, similar to the optical localizer 60, sold by Medtronic Navigation, Inc. having a place of business in Louisville, Colorado. Further alternative tracking systems are disclosed in U.S. Pat. No. 5,983,126, issued Nov. 9, 1999, which is hereby incorporated by reference. Tracking systems alterative or in addition to EM and optical tracking systems may include an acoustic, radiation, radar, etc. tracking or navigation systems.

Briefly, to be discussed in further detail herein, the imaging system 20 can include a support system which may be referred to and/or include a housing or cart 100. The imaging system 20 can further include a separate image processing unit 102 that can be housed in the cart 100. The navigation system 30 can include a navigation processing unit 110 that can communicate or include a navigation memory 112. The navigation processing unit 110 can receive information, including image data, from the imaging system 20 and tracking information from the tracking system, including the respective tracking devices 70, 72, and 74 and the localizers 60, 62. Image data can be displayed as an image 114 on the display device 64 of a workstation or other computer system 116. The image data may be processed into an image, such as a 3D reconstruction based on the image data, or displayed as raw image data, such as 2D projections. The workstation 116 can include appropriate input devices, such as a keyboard 118. It will be understood that other appropriate input devices can be included, such as a mouse, a foot pedal or the like.

The image processing unit 102 may be configured, if provided, to process image data from the imaging system 20 and transmit the image data to the navigation processor 110. It will be further understood, however, that the imaging system 20 need not perform any image processing and the image processing unit 102 can transmit the image data directly to the navigation processing unit 110. Accordingly, the navigation system 30 may include or operate with a single or multiple processing centers or units that can access single or multiple memory systems based upon system design. It is understood, however, that all of the processing units discussed herein may be generally processors that are executing instructions recalled form a selected memory, have onboard memory, or be application specific processors. Further, each of the processors may be provided or configured to perform all processing tasks discussed herein. Thus, although a specific process may be discussed as an imaging process, the navigation processing unit 110 may also be configured to perform the process.

The imaging system 20, as discussed herein, may move relative to the patient 40. The patient 40 may be fixed to an operating table or support table 120, but is not required to be fixed to the table 120. The table 120 can include a plurality of straps 124. The straps 124 can be secured around the patient 40 to fix the patient 40 relative to the table 120. Various additional or alternative apparatuses may be used to position the patient 40 in a static position on the operating table 120. Examples of such patient positioning devices are set forth in U.S. Pat. App. Pub. No. 2004/0199072, published Oct. 7, 2004, (U.S. patent application Ser. No. 10/405,068 entitled “An Integrated Electromagnetic Navigation And Patient Positioning Device”, filed Apr. 1, 2003), which is hereby incorporated by reference. Other known apparatuses may include a Mayfield® clamp.

Also, the position of the patient 40 relative to the imaging system 20 can be determined by the navigation system 30 with the patient tracking device 70 and the imaging system tracking device 72. Accordingly, the position of the patient 40 relative to the imaging system 20 can be determined. An exemplary imaging system, such as the O-arm® may also be operated to know a first position and can be repositioned to the same first position within a selected tolerance. The tolerance may be about 0.01 millimeters (mm) to about 10 mm, about 0.01 mm to about 2 mm, and about 10 microns. This allows for a substantially precise placement of the imaging system 20 and precise determination of the position of the imaging device 20. Precise positioning of the imaging portion 22 is further described in U.S. Pat. Nos. 7,188,998; 7,108,421; 7,106,825; 7,001,045; and 6,940,941; all of which are incorporated herein by reference.

Physical space of and/or relative to the subject, such as the patient 40, may be referred to as subject or patient space. Image space of an image or coordinate system of an image that is generated or reconstructed with the image data from the imaging system 30 may be referred to as image space. The image space can be registered to the patient space by identifying matching points or fiducial points in the patient space and related or identical points in the image space. The imaging device 20 can be used to generate image data at a precise and known position. This can allow image data that is automatically or “inherently registered” to the patient 40 upon acquisition of the image data. Essentially, the position of the patient 40 is known precisely relative to the imaging system 20 due to the accurate positioning of the imaging system 20 in the patient space. This allows points in the image data to be known relative to points of the patient 40 because of the known precise location of the imaging system 20.

Registration may occur in various manners, such as manual or automatic registration can occur by matching fiducial points in image data with fiducial points on the patient 40. Registration of image space to patient space allows for the generation of a transformation map between the patient space and the image space. According to various embodiments, registration can occur by determining points that are substantially identical in the image space and the patient space. The identical points can include anatomical fiducial points or implanted fiducial points.

The navigation system may include one or more tracking systems that track various portions, such as tracking devices, associated with instruments. The tracking system may include a localizer that is configured to, alone or in combination with a processor, determine the pose of a tracking device in a navigation system coordinate system. Determination of the navigation system coordinate system may include those described at various references including U.S. Pat. Nos. 8,737,708; 9,737,235; 8,503,745; and 8,175,681; all incorporated herein by reference. In particular, a localizer may be able to track an object within a volume relative to the subject. The navigation volume, in which a device may be tracked may include or be referred to as the navigation coordinate system or navigation space. A determination or correlation between two coordinate systems may allow for or also be referred to as a registration between two coordinate systems.

Furthermore, image data, images generated with the image data, and/or images may be acquired of selected portions of a subject. The images may be displayed for viewing by a user, such as a surgeon. The images may have superimposed on at least a portion of the image a graphical representation (e.g., icon) of a tracked portion or member, such as an instrument. The images may have a coordinate system and define the image space. According to various embodiments, the graphical representation may be superimposed on the image at an appropriate position due to registration of an image space (also referred to as an image coordinate system) to a subject space (also referred to as an subject or physical coordinate system). A method to register a subject space defined by a subject (including a physical space relative thereto and/or inclusive of) to an image space may include those disclosed in U.S. Pat. Nos. 8,737,708; 9,737,235; 8,503,745; and 8,175,681; all incorporated herein by reference.

As discussed herein, image space to subject space registration may not be required. An imaging system (e.g., a US probe) may be tracked as may an instrument that is separate or second relative to the imaging system. Thus, the image coordinate space is tracked with the imaging system and the tracked pose of the instrument may be tracked with the same or correlated tracking system. Thus, a pose of the instrument within the image may be known based on the correlated tracking systems.

Nevertheless, image to subject registration may occur. For example, during a selected procedure, a coordinate system may be registered to the subject space or subject coordinate system due to a selected procedure, such as imaging of the subject. In various embodiments, the first coordinate system may be registered to the subject by imaging the subject with a fiducial portion that is fixed relative to the first member or system, such as the robotic system. The known position of the fiducial relative to the robotic system may be used to register the subject space relative to the robotic system due to the image of the subject including the fiducial portion. Thus, the position of the robotic system or a portion thereof, such as the end effector, may be known or determined relative to the subject. Due to registration of a second coordinate system to the robotic coordinate system may allow for tracking of additional elements not fixed to the robot relative to a position determined or tracked by the robot.

The tracking of the instrument during a procedure, such as a surgical or operative procedure, allows for navigation of the instrument during the procedure and may allow a navigated procedure. When image data is used to define an image space it can be correlated or registered to a physical space defined by a subject, such as a patient as discussed herein. According to various embodiments, therefore, the patient defines a patient space in which an instrument can be tracked and navigated. The image space defined by the image data can be registered to the patient space defined by the patient. The registration can occur with the use of fiducials that can be identified in the image data and in the patient space. The registration may be due to a determined transformation between two or more coordinate systems and the registration may occur according to any appropriate technique. Regardless, the registration may allow for a navigation relative to a subject via and image of the subject, for example illustration of a pose of an instrument relative to (e.g., superimposed on) an image of the subject.

Once registered, the navigation system 30, with and/or including the imaging system 20, can be used to perform selected procedures. Selected procedures can use the image data generated or acquired with the imaging system 20. Further, the imaging system 20 can be used to acquire image data at different times relative to a procedure. As discussed herein, image data can be acquired of the patient 40 subsequent to a selected portion of a procedure for various purposes, including confirmation of the portion of the procedure.

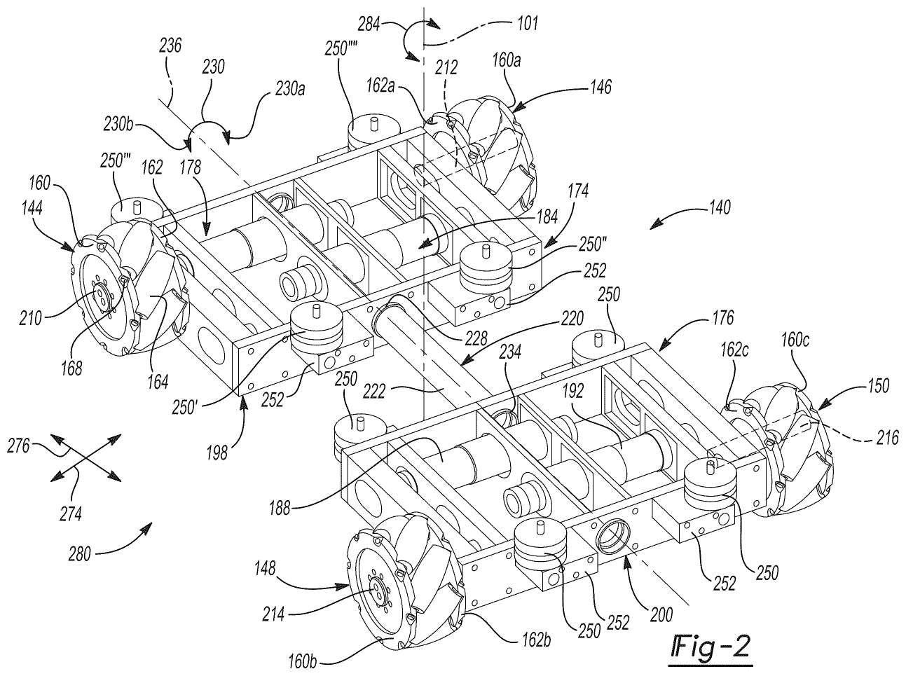

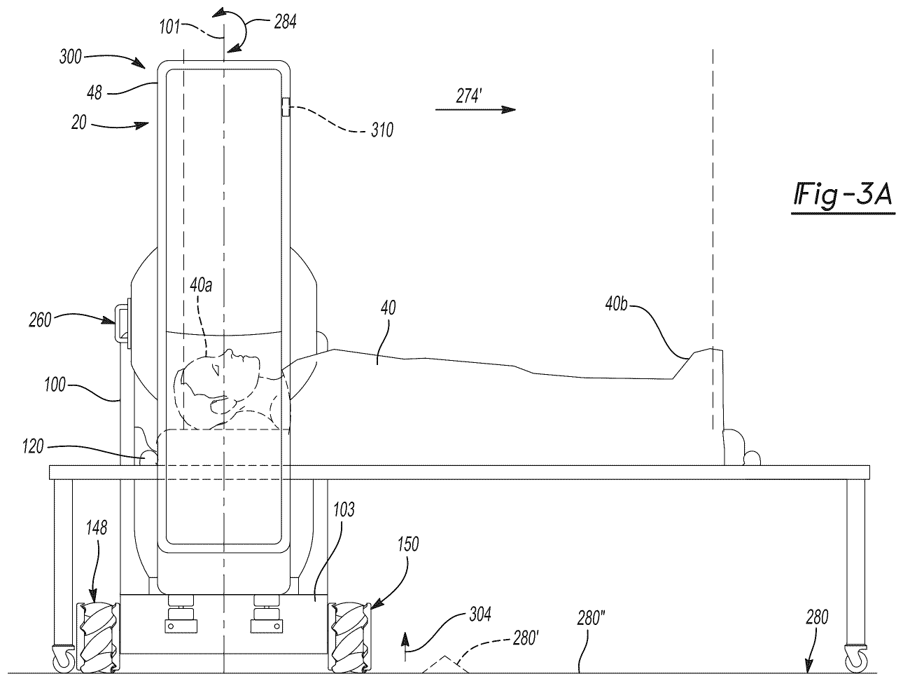

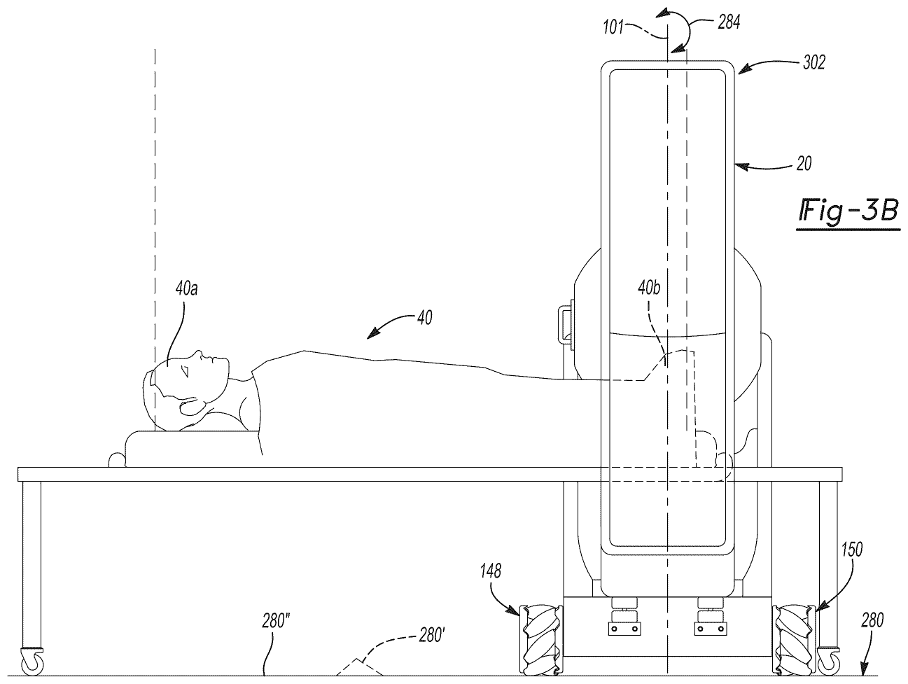

With continuing reference to FIG. 1 and additional reference to FIG. 2, FIG. 3A, and FIG. 3B, the imaging system 20 may be configured to acquire image data that is used to generate images, such as actual or virtual three dimensional images of the patient 40. As discussed above, the imaging system processor 102 and/or the navigation system processing unit 110 may be used to generate or reconstruct images for display and/or viewing by a user 69. The image data is acquired with the patient 40 placed relative to the imaging system 20 to allow the imaging system 20 to obtain image data of the patient 40. While acquiring the image data, the imaging system 20 may move relative to the patient 40.

In various embodiments, to generate a 3D image for display with the display device 64, image data can be acquired from a plurality of views or positions relative to the patient 40. The acquired image data may include a plurality of projections through the patient 40, such as those generated with x-rays, and may include 2D projections. The plurality of projections, or other appropriate image data, of the patient 40 can be used alone or with other information to generate or reconstruct an image to assist in performing a procedure on the patient 40. It is understood, however, that the patient 40 need not be the subject and other appropriate subjects may be imaged. It will also be understood that any appropriate imaging system can be used, including a magnetic resonance imaging (MRI) system, computed tomography (CT) imaging system, fluoroscopy imaging system, X-ray imaging system, etc.

To acquire the plurality of image data, including the plurality of projections of the patient, the imaging system 20 may be moved, as discussed herein. In various embodiments, the imaging system 20 includes a mobility or drive assembly or system 140 to move and/or assist in movement of the imaging system 20. The drive system 140, as discussed herein, may be a multi-directional drive system. As discussed herein, a multi-directional drive system may also be referred to as and/or refer to an omni-directional drive system including a selected wheel, such as a mecanum wheel. The multi-directional drive system may be configured to move a construct, such as the imaging system 20, in at least two directions separately and/or simultaneously. When moving, for example, the imaging system 20 may be driven by the multi-directional drive system 140 at an angle relative to two perpendicular axes. The multi-directional drive system 140 may be operated to rotate the imaging system 20 around an axis 101 defined within the imaging system 20. Moreover, the multi-directional drive system 140 may be operable to drive the imaging system 20 in a plurality of axes while acquiring image data of the subject 40. Further, in various embodiments, the drive system 140 may be operated to move the imaging system in at least two axes of motion simultaneously or separately. It is understood, however, the drive system may move the imaging system 20 in more or less than two axes simultaneously. The two axes may be in a plane, such as relative to a surface or ground 280.

The drive system 140 includes wheels or rollers, including at least one (e.g., a first) omni-directional wheel 144. The omni-directional wheel 144, which may include rollers, may translate in a plane and rotate around an axis perpendicular to the plane. During translation, the omni-directional wheel 144 may generally move in any direction from a starting point. Further, the translation and rotation of the omni-directional wheel may be substantially precise and controlled. It is understood that the drive assembly 140 may include more than the omni-directional wheel 144 and may include at least three or more omni-directional wheels. Each of the multiple wheels may be positioned at selected locations relative to one another to be driven to achieve a selected movement of the imaging system 20.

Each of the omni-directional wheels may be substantially similar, however, and include similar or identical portions. The wheels, therefore, may include a second omni-directional wheel 146, a third omni-directional wheel 148 and a fourth omni-directional wheel 150. The omni-directional wheels 144, 146, 148, 150 may include portions of any appropriate omni-directional wheels such as the heavy duty Mecanum Wheel (Item number NM254 AL. manufactured by Omni Mechanical Technology, No. 3 Yaxin Alley, Xiao Bian ST, Chang'an Town, Dongguan City, Guang Dong Province, China). As discussed herein, however, the wheels may have selected portions or features. Further, the drive system 140 may include more than one type of wheel, such as two types of wheels. Further, the drive system may include any appropriate number of wheels, such as four total wheels, but also 2, 6, or any appropriate integer. Further, not all of the wheels of the drive system need be omni-directional wheels or wheels (e.g., a track portion may be provided as part of the drive system 140).

The use of omni-directional wheels may include operation to drive one or more in a selected manner to move the imaging system 20. For example, two pairs of the wheels could be positioned at corners of a diamond relative to the base 103. One pair could be driven to move the imaging system in a first direction and the other pair could be driven to move the imaging system 20 substantially orthogonal to the first direction. Alternatively, one of each pair could be driven to rotate the imaging system 20, such as around the axis 101. Accordingly, one skilled in the art will understand that the imaging system 20 may be moved in a selected manner by selectively driving one or more of the omni-directional wheels. As discussed herein, the driving of the wheels 144-150 may be used to achieve a selected image data acquisition of the patient 40.

The omni-directional wheels 144, 146, 148, 150, with reference to FIG. 2, including the first omni-directional wheel 144 may include a plurality of components. For example, the omni-directional wheel 144 may include two external plates, including a first external plate 160 and a second external plate 162. A roller 164 and/or a plurality of rollers may be positioned at an angle relative to a plane of the plate 160 and/or the second plate 162. The roller 164 may be provided to rotate on an axle 168 that is fixedly or rotatably connected to the two end plates 160, 162. In various embodiments, the axles 168 is fixed and the roller 164 rotates around the axles 168.

With continuing reference to FIG. 2 the drive assembly 140 may be formed or provided as two drive sub-assemblies, including a first drive sub-assembly 174 and a second drive sub-assembly 176. Each of the drive sub-assemblies 174, 176 may be substantially identical, but may be controlled substantially or relatively independently, as discussed herein, to move the imaging system 20. In particular, each of the drive sub-assemblies may include two of the omni-directional wheels 144, 146, 148, 150. For example, the second drive sub-assembly 176 may include the third omni-directional wheel 148 and the fourth omni-directional wheel 150.

Each of the omni-directional wheels 144, 146, 148, 150 may be driven by a respective individual motor including a first motor 178 to drive the first omni-directional wheel 144, a second motor 184 to drive the second omni-directional wheel 146, a third motor 188 to drive the third omni-directional I wheel 148, and a fourth motor 192 to drive the fourth omni-directional wheel 150. The respective motors 178-192 may directly drive their respective omni-directional wheels 144-150, according to various embodiments. It is understood, however, that each of the respective motors 178-192 may indirectly drive their respective wheels 144-150. In various embodiments, as illustrated in FIG. 2, a belt drive or chain drive, or other appropriate indirect drive, may interconnect the motors 178-192 with the respective omni-directional wheels 144-150. It is understood that various other interconnection portions may include tensioners, pulleys, and the like to drive the respective omni-directional wheels 144-150 from the respective motors 178-192. It is further understood, however, that the wheels may be drive by direct or indirect drive from the respective motors. Further, a disconnect between the wheels 144-150 and the respective motors 178-192 may be provided, as discussed herein.

Each of the omni-directional wheels 144, as noted above, includes the outer plates 160, 162, as illustrated with respect to the first omni-directional wheel 144. Each of the other wheels 146-150 may include respective outer plates referenced by similar reference numerals augmented by lower case letters.

According to various embodiments, each of the drive sub-assemblies 174, 176 include a framework or frame structure such as a first frame 198 of the first drive sub-assembly 174 and a second frame 200 of the second drive sub-assembly 176. Each of the frames 198, 200 may be formed as a single member or multiple members connected together. Further, the frames 198, 200 may include substantially only internal cross-supports and/or a skeletonized framework with no external boundary members. The frames 198, 200 hold the respective drive mechanisms, such as the respective motors 178-192 and the various direct and indirect drive members for the respective omni-directional wheels 144-150.

In various embodiments, a wheel spindle (which may also be referred to as an axle or hub herein) 210 extends entirely or partially through the first omni-directional wheel 144 and interconnects with the frame 198. The spindle 210 is connected to the omni-directional wheel 144 to allow rotation of the omni-directional wheel 144 relative to the frames 198, 200. For example, the spindle 210 may be connected to either or both of the end plates 160, 162 to rotate the omni-directional wheel 144 when driven by the motor 178. In various embodiments an end cap may connect the spindle to the respective wheel, also various friction or fixation mechanisms may be used. Rotating the spindle 210 rotates the outer plates 160, 162 to provide motive force to the omni-directional wheel 144.

Each of the other wheels 146-150 may include respective spindles or axles such as a second spindle 212 for the second omni-directional wheel, a third spindle 214 of the third omni-directional wheel 148 and a fourth spindle 216 of the fourth wheel 150. Each of the respective spindles 210-216 may be driven by the respective motors 178-192 with the respective drive or transmission mechanisms, as discussed above. Driving of the omni-directional wheels 144-150 in a selected manner, as discussed further herein, may move the imaging system 20.

The drive assembly 140, including the first drive sub-assembly 174 and the second drive sub-assembly 176 may be interconnected with a linkage 220. The linkage 220 may be any appropriate linkage, such as a rigid member, including a rigid tubular and/or solid cylindrical member, or other appropriate linkage member. In various embodiments, the linkage 220 includes a substantially cylindrical configuration having an exterior curved surface 222. The linkage 220 may be movably connected to the respective drive sub-assemblies 174, 176 at the respective frames 198, 200.

For example, the first frame 198 may include a linkage connection 228. The linkage connection 228 may include a bearing or bushing that allows the linkage 220 to move relative to the framework 198 or the framework 198 relative to the linkage 220. In various embodiments, the framework 198 may rotate around the linkage 220 generally in the direction of double-headed arrow 230. Similarly the linkage 220 may be connected to the second drive sub-assembly 176 at a second linkage connection 234 of the frame 200. Again the linkage 220 and/or the frame assembly 220 may generally move in the direction of double-headed arrow 230 relative to one another via a pushing and/or bearing at the linkage connection 234.

Accordingly, the drive sub-assembly 174 may move relative to the second drive sub-assembly 176. In other words, the second drive sub-assembly 176 may move in a first direction, such as in the direction of arrowhead 230a, while the first drive sub-assembly 174 moves in the direction of arrowhead 230b. The linkage 220, therefore, and the respective moveable connections 228, 234 allow for the drive sub-assemblies 174, 176 to move independently relative to one another generally around the linkage 220 and an axis 236. As discussed herein, this movement of the drive sub-assemblies 174, 176 relative to one another allows for a selected movement of the imaging system 20 for acquiring image data of the patient 40. This may also, as also discussed further herein, eliminate or reduce vibration or motion of the imaging system 20 relative to the patient 40 during the acquisition of the image data.

One or more shock absorbers (also referred to as cushioning or soft mounts) 250 may be provided to connect to the imaging system 20 to the drive assembly 140. For example, a plurality of the mounts 250 may be provided on the drive assembly 140 either on the frames 198 or projections 250. The projections may be integral or added with an outer surface of a frame member of the frame 198, 200. It is understood that the platforms 252 may be formed with the frames 198, 200 and need not be a separate member, and are illustrated and discussed herein as separate components merely for illustration. Accordingly, the mounts 250 may be provided at any appropriate location of the drive assembly 140 to connect with the imaging system 20.

In various embodiments the mounts 250 may be formed of a resilient and/or compliant material, such as a natural or synthetic rubber, metal spring coil, hydraulic shock absorber, or the like. The mounts 250 allow for cushioning and reduction of vibration or elimination of vibration during movement of the drive assembly 140 relative to the imaging system 20. Accordingly, the drive assembly 140 may move over a surface 280, such as a floor, pavement, or the like while being non-rigidly connected to the imaging system 20. The mounts 250 allow for a reduction of a force directed or transferred to the imaging system 252 such as by damping or absorbing a force or motion experienced by the drive assembly 140 during motion of the drive assembly 140. The motion may be due to an inconsistency or an unevenness over which the surface 280 of the drive assembly 140 moves or may move due to motion of the drive assembly 140. For example, one or more of the motors 178-192 may cause a shocking or jerking motion during an operation which may be absorbed by the respective mount 250 to thereby eliminate or reduce motion affecting the imaging system 200. The mounts 250 may move or compress a selected amount to allow for suspension of the imaging system 20 in a substantially flat manner during imaging, such as movement of the imaging system 20 during imaging. In various embodiments, however, it is understood that the drive assembly 140 may be rigidly connected to the cart 100. Further, the linkage 220 may not be required between the drive sub-systems 174, 176.

Nevertheless, in various embodiments, the mounts 250 provide articulation about the linkage 220. For example, with discussion relative to the first sub-assembly 174, only two of the mounts 250′ and 250″ are compliant and the other two mounts 250″ and 250″ are rigidly attached. In this example, the wheels 144, 146 float and this creates a three point contact (the left rear, the right rear and the floating front assembly). Since three points define a plane, the base 103 remains relatively stationary relative to the floor 280. In another example, all of the mounts 250 are compliant and the sub-assembly 174 in total acts similar to an independent suspension to adapt to the terrain and maintain the imaging system 20 relative stationary relative to the floor 280.

Returning reference to FIG. 1 in various embodiments, a handle or manipulation assembly 260 is connected with at least a portion, such as a housing or the mobile cart 100 to move the imaging system 20. The user 69 may engage the handle assembly 260 that includes a grasping portion 262 and a sensing portion 264. The handle portion 262 may be connected with one or more sensors in the sensing portion 264 to sense a force, such as an amount of force and a direction of force applied to the handle 262. Other appropriate sensors may be included, such as a flexure, pressure sensor, or the like. In addition, other controls may be provided at the handle assembly 260. The handle assembly 260 may include portions similar to those included in the O-arm® imaging system sold by Medtronic, Inc. and/or those disclosed in U.S. Pat. No. 9,883,843 or U.S. Pat. No. 11,344,268, both incorporated herein by reference.

In various embodiments, the image processing unit 102 and/or a separate motion controller 268 may receive the signals based on a selected imaging input, such as from the user 69 or automatically to move the imaging system 20 such as with the drive system 140. The motion controller 268 may generate a drive signal to drive one or more of the motors 178-192. The motion controller 268 may be any appropriate motion controller, such as multi-axis motion controllers including Ethernet or computer card (PCI) controllers including the DMC-18×6 motion controller sold by Galil Motion Control, having a place of business in Rockland, California.

The motion controller 268, however, may be any appropriate motion controller, and may control the operation of the motors 178-192 to drive the respective wheels 144-150. By controlling the respective motors 178-192, the respective omni-directional wheels 144-150 may be rotated around the respective spindles 210-216 in an appropriate manner. By driving the omni-directional wheels 144-150 around the respective spindles 210-216 in a selected, manner the imaging system 20 may be moved in or along selected and/or appropriate axes.

Movement of the imaging system, such as with the handle assembly 260, may be fast or course and/or slow or fine. During course movement the imaging system 20 may be moved rapidly, such as about 0.9 miles per hour (MPH) to about 2.0 MPH, including about 1.5 MPH based on a measured or sensed force applied by the user 69. During fine movement, the imaging system 20 may be moved more slowly, such as about 0.01 MPH to about 0.3 MPH, including about 0.15 MPH based on a measured or sensed force applied by the user 69. The imaging system 20 may be moved in at least three axes by driving the omni-directional wheels 144-150 according to an appropriate control scheme. Appropriate control schemes include those that drive one or more of the wheels 144-150 to move the imaging system 20 in a selected manner such as along a selected axis of motion and in multiple axes of motion. Driving the wheels 144-150 may also be done in a closed or open loop with or without feedback form the wheels.

Driving the omni-directional wheels at different speeds and/or directions may cause different total movement of the imaging system 20. Accordingly, the imaging system 20 may be moved in a first axis 274. The first axis 274 may be an axis that is generally along a long axis of the subject, such as the patient 40. Additionally, the motion controller 268 may operate the motors 178-192 to move the imaging assembly 20 in a second axis 276, which may be substantially perpendicular to the first axis 274. The two axes 274, 276 may allow movement of the imaging system 20 generally in a plane.

The movement plane defined by the axes 274, 276 may be substantially parallel to or defined by the surface 280 on which the imaging system 20 is placed. Further the imaging system 20 may rotate around the axis 101 defined relative to the imaging system 20 and/or relative to a parallel axis 282, which may be substantially perpendicular to the first axis 274 and the second axis 276. Generally the imaging system 20 may rotate in the direction of arrow 284 around the axis 282. Further the imaging system 20 including the gantry 48 may move in the direction of the axis 282 which is substantially perpendicular to the axes 274, 276. Further, the gantry 48 may move in the direction of axis 282 and this movement may not be movement due to the drive assembly 140, although the motion controller 268 may be used to move the gantry 48 also in the direction of the axis 282.

Movement of the imaging system 20 may be performed during operation of the imaging system 20, such as gathering image data of the patient 40. The movement may be substantially automatic, such as controlled based on a selected image acquisition. The imaging system 20 may move with the drive system 140 to acquire image data of a length of the subject 40. Also, the movement may be manual, such as by movement of the imaging system 20 by the user via inputs with the handle 260. Also, the imaging system 20 may be moved while not imaging, such as to move the imaging system 20 from a first gross location (e.g. a storage locker) to a second gross location (e.g. an operating room). Therefore, the second handle assembly 290 may be limited in movement of the imaging system 20 generally along the axes 274, 276 and in the direction of arrow 284.

With continuing reference to FIGS. 1 and 2 and additional reference to FIGS. 3A and 3B, the imaging system 20 is movable relative to the patient 40. As illustrated in FIGS. 3A and 3B the patient 40 is positioned on the support 120, such as a hospital bed or operating room (e.g., radiolucent) bed. It is understood that patient 40 may be positioned at any appropriate location or room. Nevertheless, the imaging system 20 may move relative to the patient 40 to acquire image data for generation of the image 114 to be displayed on the display device 64, or any other appropriate display device.

As illustrated in FIG. 3A, the imaging system 20 may be positioned at an initial or starting position or location 300 relative to the patient 40. During operation and movement of the imaging system 20, the patient 40 need not move, according to various embodiments. The imaging system 20 may be moved relative to the subject in any appropriate manner or direction, or combination of direction to movements including those discussed above. For example, the imaging system 20 may move along axis 274 in the direction of arrow 274′, as illustrated in FIG. 3A. Accordingly, the imaging system 20 may move from near a head 40a of the patient 40 towards a foot 40b of the patient 40, as illustrated in FIG. 3B.

During movement of the imaging system 20 the gantry 48 may move in the direction of arrow 274′ and/or the entire imaging system assembly may move in the direction of arrow 274′. During movement of the entire imaging system 20, including the gantry 48 and the cart 100, the motion controller 268 may operate the drive assembly 140, including the omni-directional wheels 148, 150 to move the imaging system 20 generally in the direction of arrow 274′. The imaging system 20 may include various portions, such as those discussed above, which may also rotate around a patient 40, such as around a long axis of the patient.

As the imaging system 20 moves in the direction of arrow 274′, including the cart 100, the omni-directional wheels of the drive system 140 may rotate to move the imaging system 20 in the direction of arrow 274′. The imaging system 220 moves along the surface 280, which may also support the patient support 120. It is understood that the surface 280 may be substantially smooth and planar for movement of the imaging system 20. It is further understood, however, that surface 280 may include imperfections, such as bumps or projections 280′ that extend above a lower or flat portion 280″ of the surface 280. Similar low spots or depressions (not illustrated) may be present).

During movement of the imaging system 20 from the starting position 300, illustrated in FIG. 3A, to an ending or a second position 302, illustrated in FIG. 3B, the imaging system 20 may encounter the bump 280′. The bump 280′ may move one or more of the omni-directional wheels 144-150 generally in a direction away from the flat surface portion 280″, such as in the direction of arrow 304. During movement of the selected omni-directional wheel, such as the omni-directional wheel 150, the orientation or position of various portions of the imaging system, such as the detector 54 may move relative to the patient 40 from an intended path, such as one generally along a plane and defined by the axes 274, 276. The resultant image data may, therefore, not be aligned with all image frames or acquisitions of the image data acquired with the imaging system 20.

The imaging system 20 may include additional sensors such as those included in a position measurement sensor, which may be referred to as an inertial measurement unit (IMU) 310. The IMU 310 may include one or more accelerometers and one or more gyroscopes. The IMU 310 may be incorporated into various portions of the imaging system 20, such as the detector 54, the gantry 48, the imaging system support base 103 of the cart 100, or other appropriate locations.

The imaging system 20 may be moved as noted above with a selected drive system 140. The drive system may include one or more wheels of a selected type, such as a mecanum wheel. Further, it is understood that the drive assembly according various embodiments, including the drive assembly 140 and/or the drive assembly 340, may be included with other types of wheels or omnidirectional wheels, such as those discussed above. The types of wheels may include mecanum wheels and/or Rotacaster® omnidirectional wheels sold by Rotacaster Wheel Limited having a place of business in Tighes Hill, Australia.

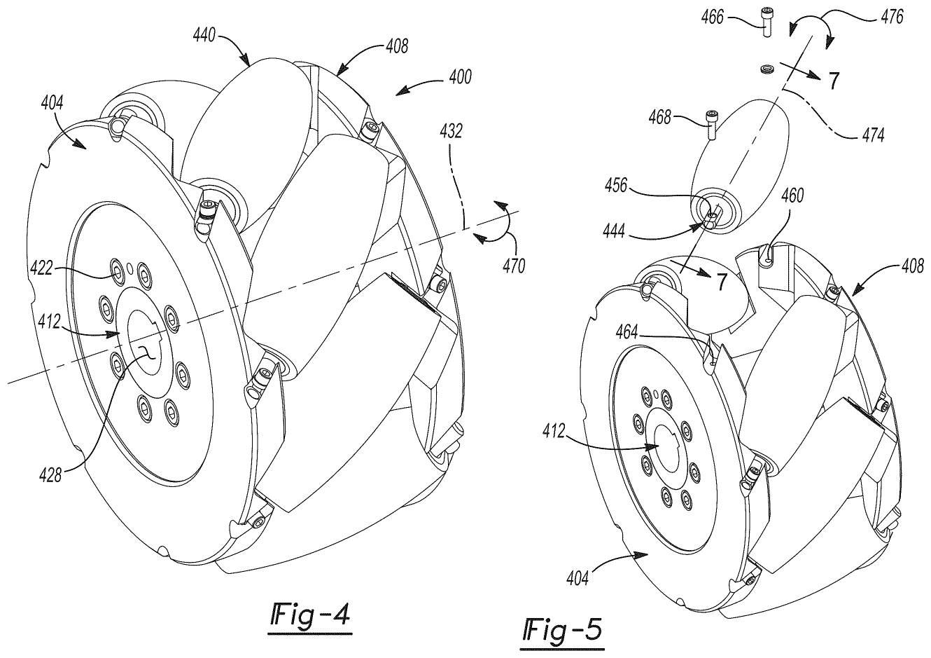

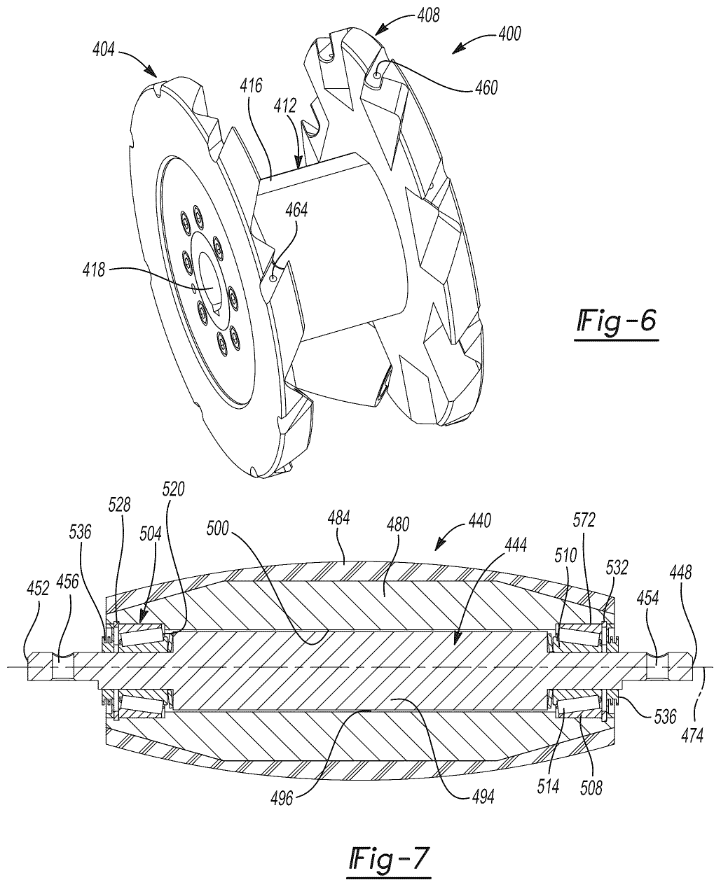

According to various embodiments, the drive system 140 may include one or more of the omni-directional wheels 144-150, as discussed above. Further, as discussed above, the omni-directional wheels may include various features that may be and/or replaced amongst any one or more of the omni-directional wheels. Accordingly, an omni-directional wheel 400, according to various embodiments, as illustrated in FIG. 4 may be used as any one or more of the omni-directional wheels 144-150, discussed above. Therefore, discussion of the omni-directional wheel 400 may be understood to relate to the drive system 140 or any appropriate drive system for the imaging system 20. The omni-directional wheel 400 may include the various components, as discussed herein and/or may include components, as discussed above, in various other omni-directional wheels and vice versa. Also, more than one of the omni-directional wheels 400 may be used in the drive system to drive a system, such as the imaging system 20. Therefore, the discussion of the omni-directional wheel 400 herein is understood to relate to the system discussed above and in the appropriate drive system, such as for the imaging system 20.

According to various embodiments, the omni-directional wheel 400 may include a first wheel side plate 404 and a second wheel side plate 408. The wheel side plates 404, 408 may be designed in any appropriate manner, as is understood by one skilled in the art. For example, the side plates may include respective bores or holes to allow placement and/or engagement with a wheel hub or spindle 412. The wheel spindle 412 may be fitted to the side plates 404, 408 in any appropriate manner. For example, the wheel spindle 412 may include an annular wall thickness or cylindrical wall between an outer surface 416 and an inner surface 418. Into the thickness, one or more fasteners 422 may be passed through the respective wheel plates 404, 408 to engage the wheel spindle 412. Thus, the wheel spindle 412 may be fixed relative to the wheel plates 404, 408. According to various embodiments, therefore, the wheel spindle 412 may be rotationally fixed or removably fixed relative to the wheel plates 404, 408.

The wheel spindle 412, however, may define a central passage or bore 428 that may extend along a rotation axis 432 defined through the wheel spindle 412. The passage 428 may engage a driveshaft of a motor, as discussed above, of the drive system 140. Thus, the wheel 400 may be driven in an appropriate manner, such as that discussed above.

The wheel 400 may include one or more roller assemblies 440. According to various embodiments, the wheel 400 may include eight of the roller assemblies 440. It is understood, however, that the wheel 400 may include any appropriate number of the wheel assemblies and eight is merely exemplary. Generally, the wheel assembly 400 includes an appropriate whole number of the roller assemblies 440, such as between 1 and 100, including between 2 and 20, and further including between 4 and 12. Nevertheless, it is understood that the wheel assembly 400 may include any appropriate number of the roller assemblies 440.

The roller assemblies 440 may include various components, as discussed further herein. Generally the roller assemblies 440 are connected to the wheel assembly 400, such as to one or both of the side plates 404, 408. According to various embodiments, for example, the roller assemblies 440 may include a roller assembly axle 444 (FIG. 7). The roller assembly axle 444 may extend between a first terminal end 448 and a second terminal end 452. Formed through the axle 444 near the respective terminal ends 448, 452 may be one or more connection bores 454, 456. The connection bores may be used to connect the axle 444 to the respective side plates 404, 408. According to various embodiments, for example, the side plates 404,408 may include receiving bores 460 and 464. One or more selected fasteners, such as a first fastener 466 and a second fastener 468 may pass through the respective bores 454, 456 of the axle 444 and be engaged into the bores 460, 464 of the respective side plates 404, 408. The fasteners 466, 468 may be any appropriate fasteners such as wheel studs, threaded screws, bolts, or the like. The fasteners 466, 468 may hold the axle 444 substantially fixed relative to the side plates 404, 408. As discussed further herein, however, various portions of the roller assembly 440 may rotate relative to the axle 444 that is fixed relative to the side plates 404, 408. The fasteners 466, 468 may, therefore, be accessed at an outer edge of the respective side plates 404, 408.

The wheel assembly 400 may rotate around the central axis 432 formed through the wheel spindle 412 in any appropriate direction, such as in the direction of the double headed arrow 470. The rollers, 440, due to the fixing of the axle 444 to the respective side plates 404, 408 may also rotate around an axis 474 defined by a longitudinal axis of the roller axle 444. Generally, the rollers 440 may rotate in the direction of the double headed arrow 476. According to various embodiments, the rollers 440 may rotate substantially freely around the roller axle 444 of the roller assembly 440. In various embodiments, for example, the roller assemblies 440 may include a roller core 480 and a roller tread 484 that together or each portion individually may form a rotating portion 490 that may rotate substantially freely relative to, such as around, the roller axle 444. As discussed further herein, therefore, the wheel 400 may rotate around the axis 432 generally in the direction of the double headed arrow 470 and the roller assemblies 440, including at least a roller portion thereof including one or more of the core 480 and the tread 484 may rotate around the axis 474 defined by the axle 440 such as in the direction of the double headed arrow 476.

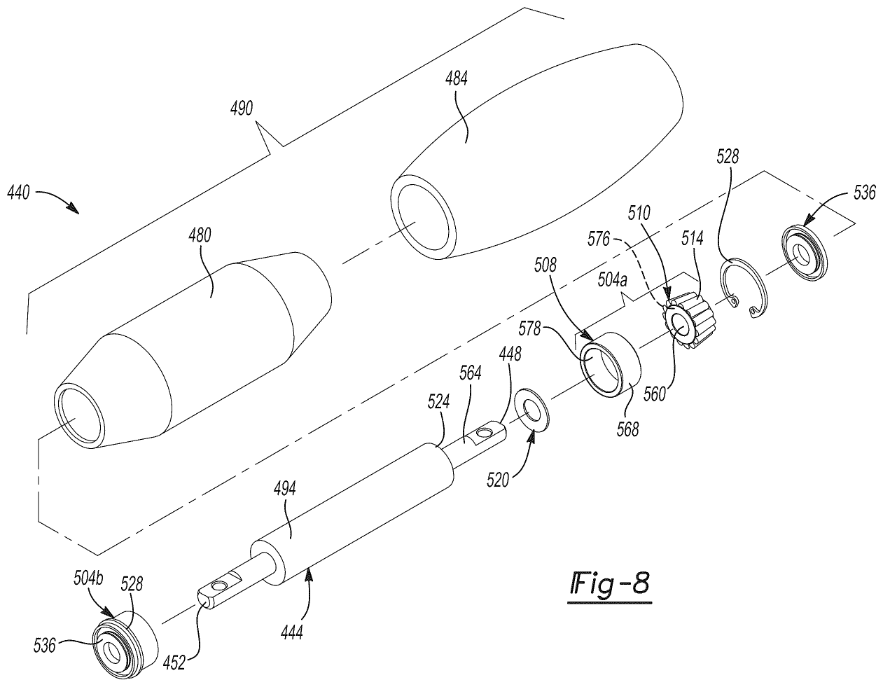

Operation of the roller assembly 440 will be described in greater detail relative to the portions of the roller assembly 440. Turning reference to FIG. 7 and FIG. 8, the roller assembly 440 may include various components. The various components may be assembled into the roller assembly 440. The roller assembly 440, therefore, may be preassembled or assembled at any appropriate time. The roller assembly 440 may then be connected to portions to form the wheel 400, such as the respective side plates 404, 408 at any appropriate time. The wheel assembly 400, therefore, may be assembled with various components including one or more of the roller assemblies 440, the side plates 404 and 408, the wheel spindle 412, and the respective fasteners 422. The roller assemblies 440 need not be assembled with the other components of the wheel 400, but may be assembled separately and independently thereof.

The roller assembly 440 may include the tread portion 484 and the core portion 480. The tread 484 and the core portion 480 may form or define the rolling portion 490 of the roller assembly 440. The rolling portion 490 may rotate relative to the axle 444. The axle 444 may include a central or middle portion 494 that is positioned between the terminal ends 448 and 452. The central portion 494 may be positioned within the core 480, but may not engage the core 480, such as having a gap 486 that is formed or defined between the central portion 494 and the core 480. The core 480 may define a central bore or passage 500 through which the axle 444 may pass and also include portions to engage or hold a bearing assembly 504 and/or other components.

Briefly, the bearing assembly 504 may include an outer race 508, and inner race or cage 510, and one or more bearing rollers 514. The bearing rollers 514 may be positioned between the outer race 508 and the inner race 510. The bearing rollers may be positioned at an angle or taper, as discussed further herein. Thus, the bearing assembly, according to various embodiments, may be a taper or tapered bearing. The bearing rollers may have a selected diameter, such as about 0.1 mm to about 10 mm, including about 1 mm to about 8 mm, including about 2 mm to about 6 mm.

In the roller assembly, the bearing assembly 504 may be preloaded with a preloading member, such as a spring 520 that may be a Bellville spring. The spring 520 may engage an end 524 of the central portion 494 of the axle 444. An engaging or holding member 528 may include at least one of a clip ring, clamp, C-clamp, or a c-spring. The retainer 528 may engage or hold the bearing assembly 504 within the roller assembly 440. The retainer 528 may be held within a groove or depression 532 formed within the core 480. According to various embodiments, for example, the preload member 520 may engage the bearing assembly 504 and apply a pressure or a preload to the bearing assembly 504 and against the retainer 528.

The roller assembly 440 may further include a protective assembly or member 536. According to various embodiments, the protective member 536 may be a shield or a seal. The protective member 536 may protect an internal portion of the roller assembly, such as the bore 500 and/or the bearing assembly 504. As is generally understood in the art, a shield may include a gap or opening around the axle 444 to allow rotation of portions, such as the rotating portion 490, relative to the axle 444. Nevertheless, the shield may eliminate or limit ingress of material into the passage and/or the bearing assembly 504. Similarly, the shield may limit or eliminate egress of a lubricant, such as grease, from the bearing assembly 504. A seal may contact both of the roller core 508 and the axle 444. For example, the shield as the protective member 536 may include a retaining ring or member to fix it relative to the core 480 and a resilient portion, such as a selected natural or synthetic rubber portion that engages the axle 444. Therefore, the sealing member may rotate relative to the axle 444, such as rotating with the core 480, while maintaining contact with the axle 444. The seal may also substantially limit or eliminate ingress of materials, such as dust and debris, and egress of other materials such as a lubricant, including grease. The seal, however, may limit to a greater degree than the shield ingress and egress due to the constant contact with the axle and the core even during rotation of the rotating portion 490.

With continued reference to FIGS. 7 and 8, the roller assembly 440 may include two of the bearing assemblies 504, which may be referred to as 504a and 504b. Similarly, the roller assembly 440 may include two of the preload members 520, two of the retaining clips 528, and two of the protective members 536. Therefore, the roller assembly 440 may be provided as a single assembly unit including the roller portion 490 (which may be inclusive of the core 480 and the tread 484), one or more of the bearing assemblies 504, one or more of the preloaded portions 520, one or more of the retainer clips 528, one or more of the protective portions 536, and the axle 444. This may allow the roller assembly 440 to be assembled with the selected portions, as noted above, into a single unit. The axle may be held within the rotating portion 490 with the bearing assembly 504, the preloaded portion 520 and the retaining clip 528. The roller assembly 440 may be protected from an external environment, at least the internal portions thereof, with the protective member 536.

The bearing assembly 504 may allow for the roller assembly 490 to rotate relative to the axle 444. As discussed above, there may be a gap 496 formed between the internal portion 494 and the core 480. Therefore, the bearing assembly 504 may bear or hold the force against the axle 444 due to contact of the tread 484 with the surface 280. The inner race 510 may include an inner surface 560 that contacts an external surface 564 of the axle 444. The contact between the inner surface 560 of the inner race 510 and the outer surface 564 of the axle 444 may allow for a substantial fixation of the inner race 510 to the axle 444. In addition or alternatively, the inner race 510 may include a selected configuration to be rotationally fixed with the axle 444. The contact may be a friction fit or other connection such that the inner race 510 is fixed to the axle 444. As discussed above, the axle 444 may also be fixed to the outer walls 404, 408 of the wheel 400. Therefore, the inner race 510 may also be fixed relative to the outer walls of the wheel 400 as it is fixed to the axle 444. In other words, the inner race 510 may not move or rotate and/or rotate about zero degrees to less than five degrees in a direction when a force is applied in that direction. It is understood by one skilled in the art, however, that the elimination of all movement may not be possible. As such, a fixed inner race 510 may be understood to move zero to degrees to less than ab out 90 degrees.

The outer race 508 may include an outer wall 568 that contacts and is fixed or substantially fixed to an inner wall 572 of the core 480. In being substantially fixed, the outer race 508 may not move or rotate and/or rotate about zero degrees to less than about five degrees in a direction when a force is applied in that direction. It is understood by one skilled in the art, however, that the elimination of all movement may not be possible. As such, a fixed outer race 508 may be understood to move zero to degrees to less than ab out 90 degrees. The outer race 508, therefore, may be fixed to the core 480. Again, a friction fit may be formed between the external wall 568 of the race 508 and the inner wall 572 of the core 480. Therefore, the outer race 508 moves with the core 480. The outer race 408, therefore, rotates with the core 480 relative to the axle 444.

Positioned between the inner race 510 and the outer race 508 is one or more roller bearings 514. The roller bearings 514 may be rotationally held relative to the inner race 510 such that the inner race 510 forms a cage and/or a cage is provided to hold the roller bearings 514. The roller bearings 514, therefore, may rotate relative to the inner race 510 and the outer race 508. For example, the inner race 510 may include an outer wall 576. The outer race 508 may define an inner wall 578. The roller bearings 504 may rotationally engage both the outer wall 576 of the inner race 510 and the inner wall 578 of the outer race 508. Therefore, the roller bearings 514 may rotate within the bearing assembly 504 as the rotating portion 490 rotates relative to the axle 444.

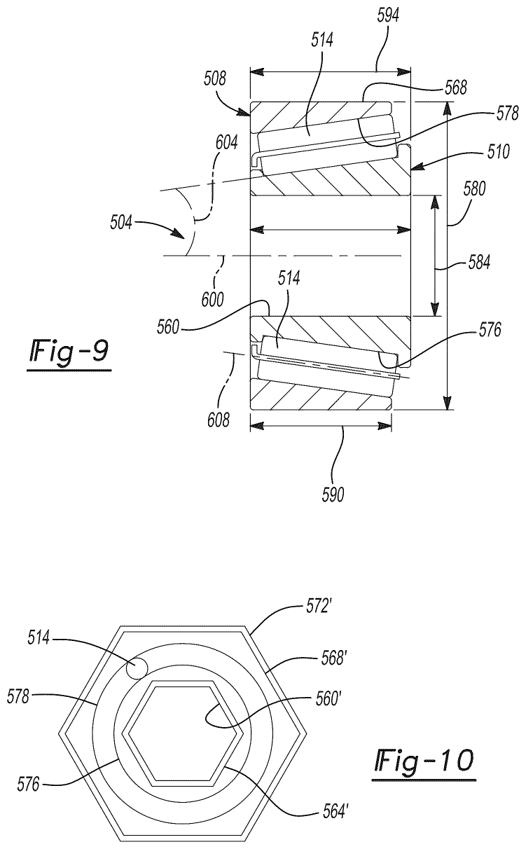

With continuing reference to FIGS. 7 and 8, an additional reference to FIG. 9, the bearing assembly 504 will be discussed in greater detail. Again, as discussed above, the roller assembly may include more than one of the bearing assemblies 504. Therefore, the discussion of the bearing assembly 504 as illustrated in FIG. 9 relates to any number of the roller bearing assemblies 504 provided in the wheel assembly 500 and/or each of the roller assemblies 540. The bearing assembly 504, as illustrated in FIG. 9, includes the outer race 508, the inner race 510, and the bearing members 514. Initially, the bearing assembly 504 may include a plurality of the roller bearings 514. As illustrated in FIG. 9, the bearing assembly is illustrated with two of the roller bearing members 514. It is understood that any appropriate number of the roller bearing members 514 may be provided with the bearing assembly 504.

The bearing assembly 504 can include various dimensions to interact with the roller assembly 540. For example, the outer race 508 may include an outer dimension 580 to engage the inner surface 572 of the core 480. The outer dimension 580 may be an outer diameter, according to various embodiments. It is understood, however, that the outer dimension 580 may be any appropriate dimensions of any appropriate geometric shape such as a hexagon, square, octagon or the like. According to various embodiments, however, the outer dimension 580 is an outer diameter and may be about 15 mm to about 40 mm, including about 15 mm to about 25 mm, and further including about 24 mm. Nevertheless, the external dimension 580 may be any appropriate dimension such as any integer or fraction thereof between about 1 mm and about 100 mm.

The inner race 510 may have an internal dimension 584 that may be defined by the internal wall 560. The internal dimension 584 may be an internal diameter. Again, however, the internal dimension 584 may be a dimension of any appropriate geometric shape such as a square, hexagon, octagon or the like. According to various embodiments, however, the internal dimension 584 may be an internal diameter that is about 5 mm to about 15 mm, including about 8 mm to about 12 mm, and further including about 9 mm to about 10 mm, and values therebetween. For example, the internal diameter may be about 9.4 mm, about 9.6 mm, about 10 mm. Further the dimensions may be understood to be values exclusive of a tolerance.

As discussed, the outer dimension 580 is provided to engage with the internal dimension or geometry of the roller such as on the internal surface 572. The internal dimension 584 of the internal surface 560 is provided to engage the external surface 564 of the axle 444. Therefore, the dimensions 580, 584 may be provided in any appropriate range or value to provide an appropriate engagement with respective portion of the roller assembly 440. If the geometries of the respective surfaces 572, 568, 560, and 564 are cylindrical, the dimensions 580, 584 may be diameters.

The respective surfaces, as noted above, may include appropriate geometries such as non-cylindrical geometries including square, hexagon, octagon, or the like. For example, with reference to FIG. 10, the various geometries may include a hexagon. Therefore, the inner surface of the 572 of the core 580 may be a hexagon such as illustrated by the hexagon 572′. The external surface of the outer race 508 may be a hexagonal geometry 568′. Similarly, the other geometries may be any appropriate geometries such as the outer geometry of the axle 444 may be a hexagon 564. Thus, the inner geometry of the outer inner race 510 may include an inner surface 560′. As discussed above, the respective geometry or surfaces are provided to engage one another and allow for substantial fixation, at least a rotational fixation, of the outer race 508 to the roller core 480 and the inner race 510 to the axle 444. Thus, the respective geometries may be any appropriate geometries including cylindrical as illustrated in FIG. 8 and/or other geometries such as hexagonal, as illustrated in FIG. 10.

It is understood that the respective geometries may be mixed and match as long as they are in concert with one another such as the hexagonal surface 572′ and 568′ between the outer race 508 and the core 480 and a cylindrical geometry between the inner race 510 and the axle 444. Therefore, one skilled in the art, will understand according to various embodiments, the respective geometries may be different and need not be identical to one another. Nevertheless, the roller bearings 514 may still be provided in a substantially cylindrical or smooth geometry surfaces 576, 578 as illustrated in FIG. 10.

Returning to FIG. 9, the bearing member 504 may further include various selected dimensions such as an outer race thickness 590 that may be about 5 mm to about 20 mm, including about 8 mm to about 15 mm, and further including about 13 mm. Further, the inner race 510 may include a thickness 594 that may be an appropriate thickness such as about 5 mm to about 20 mm, including about 8 mm to about 15 mm, and further including about 12 mm. It is understood the various dimensions may be provided in an appropriate manner, including such as about 10 mm for the internal diameter 584, according to various embodiments.

The bearing assembly 504 may define a central axis 600 that may extend through the passage defined by the inner surface 560. The central passage 560 may be substantially cylindrical or have another appropriate geometry, as discussed above, such that it is generally symmetrical around the central axis 600. Similarly, the external geometry of the bearing member 504 may be substantially symmetrical around the central axis 600, as well. Nevertheless, the inner surface 578 of the outer race 508 and the outer surface 576 of the inner race 510 may be formed at an angle 604 relative to the central axis 600. As illustrated in FIG. 9, the angle 604 may be an internal angle and may be an acute angle. The angle 604, however, may also be defined as an external angle or an obtuse angle relative to the axis 600. Further, the bearing members 514 may rotate around an axis 608 that is substantially parallel with the walls 576 and 578 of the respective races 510, 508. Therefore, the angle 604 may also be formed between the axis 608 and the axis 600.

The angle 604 forms the bearing member 504 into a tapered bearing member. Each of the rollers 514, therefore, may be positioned at an angle relative to the central axis 604. The tapered bearing member or bearing assembly 504 may be used as the bearing member 504, discussed above. The tapered bearing 504 may be understood to be the bearing assembly 504 as discussed herein according to various embodiments. The tapered bearing assembly 504 may allow forces to be encountered by the roller assembly 440, such as the tread 494 and applied to the tapered bearing assembly 504 in a selected manner to achieve a selected life and reduction of wear on the bearing assembly 504. The angle 604 may be selected based upon various geometries, such as the outer geometry of the tread 584, the mass of the assembly carried by the drive system 140 and/or each of the respective wheels, dimensions of the axle 444, or other appropriate considerations. The angle 604 may be greater than about 0.5 degrees to about 20 degrees, including about one degree to about 15 degrees, including about two degrees to about 15 degrees, and further including about five degrees to about 11 degrees. It is understood, however, that the angle 604 may be selected to be any appropriate angle such as any whole or partial angle between about 0.1 degrees to about 30 degrees.

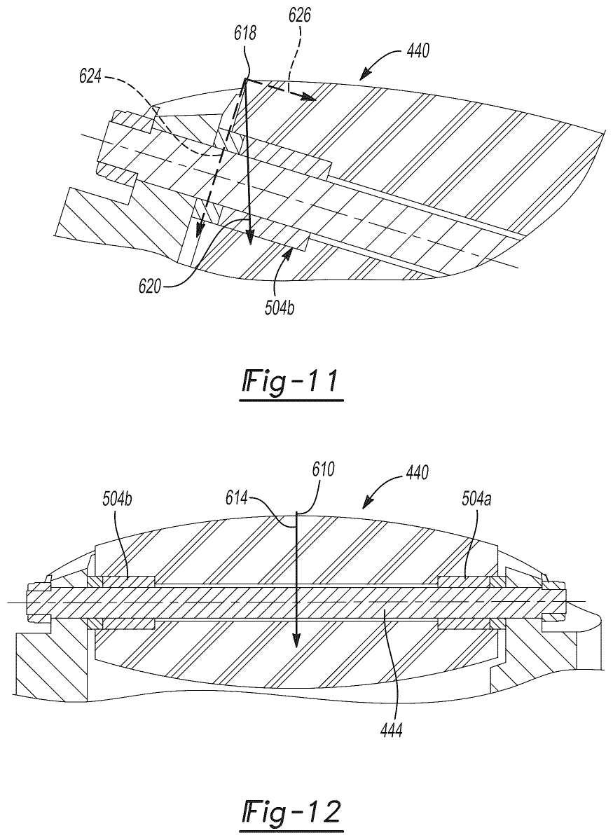

Turning reference to FIG. 11 and FIG. 12, the roller 440 of the wheel 400 may experience forces relative to various portions of the roller assembly, such as near the bearing assembly 504. As illustrated in FIG. 11, during various motions, a force may be applied generally at a center 610 of the tread and generally along a vector or arrow 614. The force applied generally in the direction of the arrow 614 may apply a force that is loaded on both of the bearing assemblies 504 at either end of the axle 444 substantially equally. The roller assembly 440, however, during movement of the drive assembly 140, may experience forces at an edge or end 618 of the roller assembly 440.

The forces that are experienced at the end may engage or apply force to one or more of the bearing assemblies, such that the bearing assembly 504b differently than to the other of the bearing assemblies. The load on one other bearing assemblies, for example, may have a general direction 620 but would have a radial component 624 and an axial component 626. Therefore, the respective components 624, 626 may act on the bearing assembly 504b in a selected manner. The tapered bearing assembly 504 may allow for maintaining or ensuring a rolling force of movement between the outer race 508 and the inner race 510 even with the load applied as illustrated in FIG. 12. Thus, the bearing assembly 504 may allow for maintaining a rolling engagement or movement between the rolling assembly 490 and the axle 444 even at loads that may not or are not at a center 610 of the roller assembly 440.

The roller assembly 440, including the roller portion 490 may be formed at selected times in selected manners. For example, the core 480 may be a cast or machined aluminum core. The tread 484 may be molded onto the core 480. The tread 484 may be formed of a selected material, such as a natural or synthetic rubber, including urethane or a urethane matrix. Appropriate materials may be provided to ensure a selected amount of traction of the wheel 400 relative to the surface 280 during a selected movement of the system, such as the imaging system 20. It is understood in various embodiments, however, that the tread 484 need not be formed separately or as a separate member or as an assembly with the core 480. In various embodiments, the tread 484 may be a single unit as the rolling portion 490 and the bearing assembly 504 may engage the tread 484 without a separate core 480.

Further, the bearing assemblies may be formed of selected materials. For example, the inner race 510, the outer race 508, and the roller bearing portions 514 may be formed of selected metals or metal alloys. In various embodiments, the bearing member 508 may be formed of selected steel or stainless steel alloys of an appropriate toughness and stiffness.

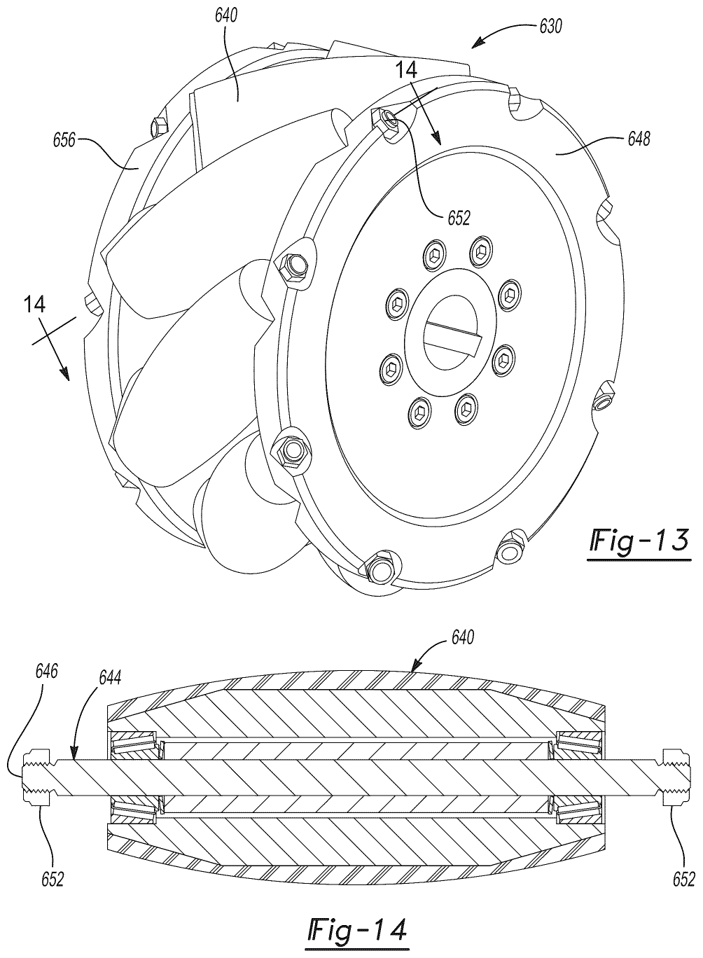

According to various embodiments, the drive assemblies 140 may include selected wheel assemblies, such as the wheel assembly 400 illustrated above. In various embodiments, however, a wheel assembly 630 may also be provided and/or alternatively be provided. The wheel assembly may include one or more roller assemblies 640. The roller assembly 640 may include components that are similar or identical to the portions of the roller assembly 440. Generally, for example, the roller assembly 640 as illustrated in FIG. 13 and FIG. 14, may be substantially identical to the roller assembly 440 save for an axle 644. The axle portion 644 may include a terminal end 646 that includes an external thread. The terminal end or an end of the axle 644 may pass through a bore in an end plate 648 of the wheel assembly 630 and a nut 652 may engage or be threaded onto the threaded portion of the axle 644. Therefore, the roller assembly 640 may pass through a portion of the wheel plate 648 and also a second wheel plate 656. The roller assembly 640, therefore, may not be dropped into the wheel assembly 630, as allowed and illustrated with the wheel assembly 400, but may be engaged or passed through the outer plates 648, 656. The roller assembly 640 may, however, be otherwise substantially identical to the roller assembly 440 as discussed above. Therefore, the roller assembly 640 may include the various portions and features as noted above. The wheel assembly 630 may be used in place of all or only some of the wheels of the drive assemblies 140 as discussed above. Nevertheless, the wheel assembly 630 may be used with the selected system.

Accordingly, as discussed above, the drive assemblies 140 of the selected system, such as the imaging system 20, may be provided to allow movement of the imaging system 20. The wheels may include appropriate wheels, such as the wheel assembly 400 and/or the wheel assembly 630. The wheel assemblies may be understood to be omni-directional or multi-directional wheels and include the roller assemblies as discussed above. The wheels may be driven in any appropriate manner and all for movement as discussed above while including the bearing assemblies 504 to assist and maintaining the roller assembly of the respective wheel assemblies. As noted above, the roller assemblies 440, 640 may allow for assembly of the roller assemblies separate from the wheel assemblies as a whole. Therefore, the wheel assemblies may be assembled with any appropriate roller assemblies to allow for an efficient manufacture and assembly of the wheel assemblies.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C#, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically disclosed otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements. The processor or processors may operate entirely automatically and/or substantially automatically. In automatic operation the processor may execute instructions based on received input and execute instructions in light thereof. Thus, various outputs may be made without further or any manual (e.g., user) input.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A system to acquire image data of a subject, comprising:
an imaging assembly comprising a detector and a gantry;
a cart having a support structure configured to support at least the gantry away from a surface; and
a multi-directional wheel assembly rotatably mounted relative to the cart;
wherein the multi-directional wheel assembly comprises a roller assembly having an axle and a tapered bearing.

2. The system of claim 1, wherein the roller assembly further comprises at least one bearing system including the tapered bearing and at least one of a seal or a shield;
wherein the at least one of the shield or the seal is operable to reduce or eliminate ingress of debris into the tapered bearing or egress of a lubricant from the tapered bearing.

3. The system of claim 2, wherein the bearing system further comprises a pre-load member;
wherein the pre-load member is configured to maintain a selected load on the tapered bearing.

4. The system of claim 3, wherein the bearing system further comprises a tapered bearing retaining member.

5. The system of claim 4, wherein the axle extends from a first end to a second end;
wherein the tapered bearing comprises a first tapered bearing and a second tapered bearing;
wherein the first tapered bearing is positioned near the first end of the axle and the second tapered bearing is positioned near the second end of the axle.

6. The system of claim 5,
wherein the bearing system comprises a first bearing retaining member and a second retaining member;
wherein the first retaining member is positioned outboard of the first tapered bearing on the axle and the second retaining member is positioned outboard of the second tapered bearing on the axle.

7. The system of claim 6, wherein the multi-directional wheel assembly comprises a plurality of the roller assemblies;
wherein each roller assembly of the plurality of roller assemblies has one of the axle, the first tapered bearing, the first bearing system, the second tapered bearing, and the second bearing system.

8. The system of claim 1, wherein the multi-directional wheel assembly comprises a plurality of the roller assemblies;
wherein each roller assembly of the plurality of roller assemblies is included as an individual assembly and has one of the axle and at least one of the tapered bearing.

9. The system of claim 1, wherein the tapered bearing comprises an inner race, an outer race, and a roller bearing member configured to roll between the inner race and the outer race.

10. The system of claim 9, wherein the tapered bearing comprises a cage configured to capture the roller bearing member between the inner race and the outer race.

11. The system of claim 9, wherein the inner race of the tapered bearing has an inner diameter of about 10 mm.

12. The system of claim 9, wherein the outer race of the tapered bearing has an outer diameter of about 24 mm.

13. The system of claim 1, further comprising:
a drive system mounted to the base;
wherein the drive system is operable to receive an input to move the cart via the multi-directional wheel assembly.

14. The system of claim 1,
wherein the detector is configured to detect an emitted energy, wherein the detection of the emitted energy is operable to generate image data;
wherein the gantry defines an enclosed annular space; and
wherein the detector is operable to move within the enclosed annular space.

15. A method for providing a mobility system for an imaging system, the method comprising:
providing an imaging assembly comprising a detector and a gantry;
providing a cart having a support structure a configured to support at least the gantry away from a surface; and
providing a multi-directional wheel assembly rotatably mounted relative to the cart;
providing the multi-directional wheel assembly with a roller assembly having an axle and a tapered bearing.

16. The method of claim 15, further comprising:
providing the roller assembly with at least one bearing system including the tapered bearing;
positioning at least one of a seal or a shield relative to the bearing system;
reducing or eliminating ingress of debris into the tapered bearing or egress of a lubricant from the tapered bearing due to the positioned at least one of the shield or the seal.

17. The method of claim 16, further comprising:
pre-loading the bearing system with a pre-load member;
wherein the pre-load member is configured to maintain a selected load on the tapered bearing.

18. The method of claim 17, further comprising providing a tapered bearing retaining member.

19. The method of claim 18, further comprising:
providing the axle to extend from a first end to a second end;
providing the tapered bearing as a first tapered bearing and a second tapered bearing;
positioning the first tapered bearing near the first end of the axle; and
positioning the second tapered bearing near the second end of the axle.

20. The method of claim 19, further comprising:
providing a first bearing retaining member and a second retaining member;
positioning the first retaining member outboard of the first tapered bearing on the axle; and
positioning the second retaining member outboard of the second tapered bearing on the axle.

21. The method of claim 20, further comprising:
providing each roller assembly of the plurality of roller assemblies with one of the axle, the first tapered bearing, the first bearing system, the second tapered bearing, and the second bearing system.

22. The method of claim 15, further comprising:
providing each of the multi-directional wheel assembly with a plurality of the roller assemblies; and
providing each roller assembly of the plurality of roller assemblies with one of the axle and at least one of the tapered bearing.

23. The method of claim 15, further comprising:
providing the tapered bearing with an inner race, an outer race, and a roller bearing member configured to roll between the inner race and the outer race.

24. The method of claim 23, wherein the inner race of the tapered bearing has an inner diameter of about 10 mm.

25. The method of claim 15, further comprising:
mounting a drive system to the base;
wherein the drive system is operable to receive an input to move the cart via the multi-directional wheel assembly.

26. The method of claim 15
providing each roller assembly of the plurality of roller assemblies as an assembled individual roller assembly including at least the axle and at least one of the tapered bearing and a at least one preload member.
